(19) United States
(12) Patent Application Publication
(10) Pub. No.: US 2024/0268897 A1
(43) Pub. Date: Aug. 15, 2024

(54) USING MACHINE LEARNING AND 3D PROJECTION TO GUIDE MEDICAL PROCEDURES

(71) Applicant: THE REGENTS OF THE UNIVERSITY OF CALIFORNIA, Oakland, CA (US)

(72) Inventors: Raj M. VYAS, Orange, CA (US); Lohrasb Ross SAYADI, Orange, CA (US)

(73) Assignee: THE REGENTS OF THE UNIVERSITY OF CALIFORNIA, Oakland, CA (US)

(21) Appl. No.: 18/568,938

(22) PCT Filed: Jun. 13, 2022
(86) PCT No.: PCT/US2022/033191

§ 371 Date: Dec. 11, 2023

Related U.S. Application Data
(60) Provisional application No. 63/209,400, filed on Jun. 11, 2021.
(60) Provisional application No. 63/210,196, filed on Jun. 14, 2021.

(51) Int. Cl. A61B 34/20; A61B 90/00; G06V 10/82

(52) U.S. Cl. CPC A61B 34/20; A61B 90/361; A61B 90/39; G06V 10/82; A61B 2034/2065; A61B 2090/366; A61B 2090/367; A61B 2090/3937; G06V 2201/03; G06V 2201/12

(57) ABSTRACT

A system for guiding a surgical or medical procedure includes a depth camera for acquiring images and/or video from a predetermined site of a subject before, during, or after a planned surgical or medical procedure and a high definition projector for projecting surgical markings onto this predetermined surgical site. The system also enables a remote educator or expert to guide the procedure by annotating a three-dimensional digital image of the subject such that this input is then projected onto the actual subject in real time. A trained machine learning guide generator is in electrical communication with the depth camera and the projector. Characteristically, the trained machine learning guide generator implements a trained machine learning model for the predetermined anatomic site. Advantageously, the trained machine learning guide generator is configured to control the projector using the trained machine learning model to bind projection such that surgical markings that guide surgical or medical procedures are stably projected onto the subject despite movement.

### CROSS-REFERENCE TO RELATED APPLICATIONS

This application claims the benefit of U.S. provisional application Ser. No. 63/210,196 filed Jun. 14, 2021 and U.S. provisional application Ser. No. 63/209,400 filed Jun. 11, 2021, the disclosures of which are hereby incorporated in their entirety by reference herein.

### TECHNICAL FIELD

In at least one aspect, the present invention relates to systems and methods for applying machine learning and 3D projection to guide surgical or medical procedures.

### BACKGROUND

Performing accurate surgery relies on a host of factors: patients' variable anatomy, their individual disease process, and of course, surgeon experience. Becoming a skilled surgeon requires years of post-graduate training in an apprenticeship model to accrue sufficient knowledge and skill. Currently, surgical or medical trainees are limited by the experience and skills of a limited number of mentors. Skill/knowledge transfer in surgery is otherwise gained through the use of books, presentations, models, cadavers, and computer simulations. The trainee still has to convert and translate the skills acquired from these sources to live subjects. As such, the process of skill transfer is slow, antiquated, and error-prone. To date, there is no system that uses artificial learning algorithms to facilitate surgical guidance and accelerate knowledge/skill transfer. In addition, no system currently exists that utilizes such AI algorithms to project relevant surgically oriented information (e.g. markings) onto the 3D anatomy of patients, thereby guiding a surgical procedure.

Accordingly, there is a need for systems performing guided surgery to facilitate skill transfer and to generally improve the quality (e.g. accuracy, etc.). of surgical and medical procedures.

### SUMMARY

In at least one aspect, a system for guiding surgical or medical procedures is provided. The system includes a depth camera for acquiring images and/or video of a predetermined surgical site from a subject during a surgical or medical procedure and a projector for projecting markings and/or remote guidance markings directly onto the predetermined surgical site during the surgical or medical procedure such that these markings and guides (i.e., the remote guidance markings) enhance procedural decision-making. In a refinement, remote guides are marking created by a remote user. A trained machine learning guide generator is in electrical communication with the depth camera and the projector. Characteristically, the trained machine learning guide generator implements a trained machine learning model for the predetermined surgical site. Advantageously, the trained machine learning guide generator is configured to control the projector using the trained machine learning model such that surgical markings are projected directly onto the subject. Advantageously, the machine learning guide generator can bind the subject's anatomy to the projected surgical markings such that the projections remain stable even with movement of the subject.

In another aspect, the system for guiding a surgical or medical procedures includes the combination of the depth camera and the projection operating in cooperation as a structured light scanner for creating three-dimensional digital images of the predetermined surgical site or another area relevant to a surgical procedure. These images are annotated and marked by a remote expert or other person and this information is projected directly onto the three-dimensional surface of the subject in real-time.

In another aspect, the system for guiding a surgical or medical procedures is configured for allowing a remote user to interact with the system. Three-dimensional digital images from structured light scans are annotated, marked, and otherwise manipulated by a remote expert or other person and this information is conveyed to the projector so that these manipulations are projected directly onto the three-dimensional surface of the subject in real-time.

In another aspect, the trained machine learning guide generator or another computing device includes a machine learning algorithm trained to identify anatomical structures identified by radiological imaging techniques such that images of underlying anatomical structures (bones, vessels, nerves, muscle, etc.) are projected along with the surgical markings onto the predetermined surgical site of the subject.

In another aspect, the trained machine learning guide generator or another computing device is configured to bind a subject's anatomy to projected surgical markings such that projections remain stable with movement of the subject.

In another aspect, the trained machine learning guide generator or another computing device is configured to bind surface anatomy captured by the depth camera to surface anatomy captured on radiographs such that applying machine learning algorithms to each identifies locations of shared surface landmarks with images of normal or pathologic underlying anatomic structures projected onto a surface of the predetermined surgical site.

In another aspect, the trained machine learning guide generator or another computing device is configured to guide sequential steps of the surgical or medical procedure by dynamically adjusting the surgical markings projected onto the subject during the surgical or medical procedure. This is made possible by capturing an updated 3D image of the surgical site with a new structured light scan or other methodology and application of different machine-learned algorithms.

In another aspect, the trained machine learning guide generator is trained by providing a first set of annotated images of a predetermined area of a subject's surface to a generic model to form a point detection model and training the trained machine learning guide generator using the point detection model with a second set of annotated images annotated with surgical annotation for each surgical marking.

In another aspect, the trained machine learning guide generator is further trained to identify surface and deep anatomical structures on radiographic imaging modalities and bind these structures to the patient's surface anatomy such that radiographic images of deeper anatomical structures are projected onto the predetermined surgical site of the subject.

In another aspect, the projector projects deep anatomy onto the predetermined surgical site of the subject that includes surface anatomy.

In another aspect, a system for guiding a surgical or medical procedure is provided. The system includes a depth camera for acquiring images and/or video from a predetermined surgical site of a subject during the surgical or medical procedure. The system further includes a projector for projecting surgical markings that guide surgical or medical procedures onto a predetermined surgical site during the surgical or medical procedure. The system also includes a trained machine learning guide generator in electrical communication with the depth camera and the projector. The trained machine learning guide generator is configured to implement a trained machine learning model for the predetermined surgical site, the trained machine learning guide generator is configured to control the projector using the trained machine learning model such that surgical markings are projected onto the subject. The trained machine learning model is trained by creating a general detection model from a first set of annotated digital images where each annotated digital image is marked or annotated a plurality of anatomic features and training the general detection model by backpropagation with a second set of annotated digital images from subjects that are surgical candidates or not surgical candidates. Each digital image includes a plurality of anthropometric markings identified by an expert surgeon. The trained machine learning model is able to recognize anatomic landmarks for both clinically normal (e.g. no cleft lip) and clinically pathologic (e.g., cleft lip) conditions.

In another aspect, a system for guiding a surgical or medical procedure is provided. The system includes a depth camera for acquiring images and/or video from a predetermined surgical site of a subject during the surgical or medical procedure and a projector for projecting markings and/or remote guidance markings onto the predetermined surgical site during the surgical or medical procedure such that these markings and guidance markings enhance procedural decision-making. This system can operate with or without the application of AI algorithms.

In another aspect, a method for guiding a surgical or medical procedure using the system set forth herein is provided. The method includes a step of acquiring images and/or video from a predetermined surgical site of a subject during a surgical or medical procedure. Markings that guide surgical or medical procedures are projected onto the predetermined surgical site during the surgical or medical procedure. The surgical markings that guide surgical or medical procedures are determined by a trained machine learning guide generator or directly added by a remote person onto the digital 3D model of the surgical site created by the structured light scan.

In another aspect, the machine learning algorithms identify a subject's surface anatomy and use this information to bind projections of surgical markings/guidance to this anatomy such that the projections remain stable with movement of the subject.

In another aspect, a surgical guidance system combines machine learning algorithms with both 3D surface projection and AR. By incorporating artificial intelligence algorithms that understand detailed human surface anatomy (including normal and abnormal anatomy), 3D projection can be targeted to a specific surgical or medical procedure (defined, in part, by specific surface anatomy), and surgical markings/guides can be designed with more objectivity and less human error.

Advantageously, a projection platform for surgical guidance includes 3 components: 1) a compute device (including machine learning algorithm described above) (2) a depth camera module (3) a projection system. This platform ingests visual data from the depth camera module and feeds it to the computing device where a variety of algorithms can be deployed to correct for optical aberrations in the depth camera, detect faces, mark anthropometric landmarks, and de-skew the output projection mask to be overlayed on the patient's body. After this layer of computation, the compute device relays the visual data on the projection system to be placed on the patient.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIG. 1. Schematic of a system for projecting surgical markings that guide surgical or medical procedures onto a subject during a surgical or medical procedure.

FIG. 2. Schematic of a projector-camera assembly for use in an operating room with the system of FIG. 1.

FIG. 3. Experimental design for the developing a cleft lip facial landmark annotation algorithm.

FIG. 4. Schematic of a system of a convolutional neural network.

FIGS. 5A and 5B. Markings that guide surgical or medical procedures for cleft surgery placed by a human expert.

FIGS. 5C and 5D. Markings that guide surgical or medical procedures for cleft surgery placed by a machine learning algorithm.

FIG. 6. Normalized mean error of the 21 facial landmarks important for designing a cleft lip repair, arranged from smallest to largest error. This error results from using 345 training/testing images of individuals with cleft lip.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numerical quantity, in a refinement, the term “less than” includes a lower non-included limit that is 5 percent of the number indicated after “less than.” A lower non-includes limit means that the numerical quantity being described is greater than the value indicated as a lower non-included limited. For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, 1 percent, or 0 percent of the number indicated after “less than.”

With respect to electrical devices, the term “connected to” means that the electrical components referred to as connected to are in electrical communication. In a refinement, “connected to” means that the electrical components referred to as connected to are directly wired to each other. In another refinement, “connected to” means that the electrical components communicate wirelessly or by a combination of wired and wirelessly connected components. In another refinement, “connected to” means that one or more additional electrical components are interposed between the electrical components referred to as connected to with an electrical signal from an originating component being processed (e.g., filtered, amplified, modulated, rectified, attenuated, summed, subtracted, etc.) before being received to the component connected thereto.

The term “electrical communication” means that an electrical signal is either directly or indirectly sent from an originating electronic device to a receiving electrical device. Indirect electrical communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electrical communication can be accomplished with wired components, wirelessly connected components, or a combination thereof.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

The term “electrical signal” refers to the electrical output from an electronic device or the electrical input to an electronic device. The electrical signal is characterized by voltage and/or current. The electrical signal can be stationary with respect to time (e.g., a DC signal) or it can vary with respect to time.

The term “electronic component” refers is any physical entity in an electronic device or system used to affect electron states, electron flow, or the electric fields associated with the electrons. Examples of electronic components include, but are not limited to, capacitors, inductors, resistors, thyristors, diodes, transistors, etc. Electronic components can be passive or active.

The term “electronic device” or “system” refers to a physical entity formed from one or more electronic components to perform a predetermined function on an electrical signal.

It should be appreciated that in any figures for electronic devices, a series of electronic components connected by lines (e.g., wires) indicates that such electronic components are in electrical communication with each other. Moreover, when lines directed connect one electronic component to another, these electronic components can be connected to each other as defined above.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in a software executable object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

Throughout this application, where publications are referenced, the disclosures of these publications in their entireties are hereby incorporated by reference into this application to more fully describe the state of the art to which this invention pertains.

### Abbreviations

“AI” means artificial intelligence.

“AR” means augmented reality.

“HRNet” means High-Resolution Net.

“UI” means user interface.

“c′ala” means cleft-side alare.

“sn” means subnasale.

“c” cphs means cleft-side crista philtri superioris.

“nc” ch means non-cleft cheilion.

“nc′ala” means non-cleft alare.

“nc′cphs” means non-cleft crista philtri superioris.

“c′c” means cleft columella.

“c′sbal” means cleft subalare.

“nc′sbal” means non-cleft side subalare.

“c′ch means cleft cheilion.

“nc′c” means non-cleft side columella.

“prn” means pronasale.

“ls” means labialis superioris.

“mcphi” means medial lip element cristra philtri inferioris (cupids bow peak).

“c′rl” means cleft-side red line.

“sto” means stomion.

“c′nt means cleft side Nordhoff's triangle.

“c′nt2” means cleft side Nordhoff's triangle.

“lcphi” means lateral cristra philtri inferioris (cupids bow peak).

“m′rl” means red line of the medial lip element.

“c′cphi” means cleft-side cristra philtri inferioris (cupids bow peak).

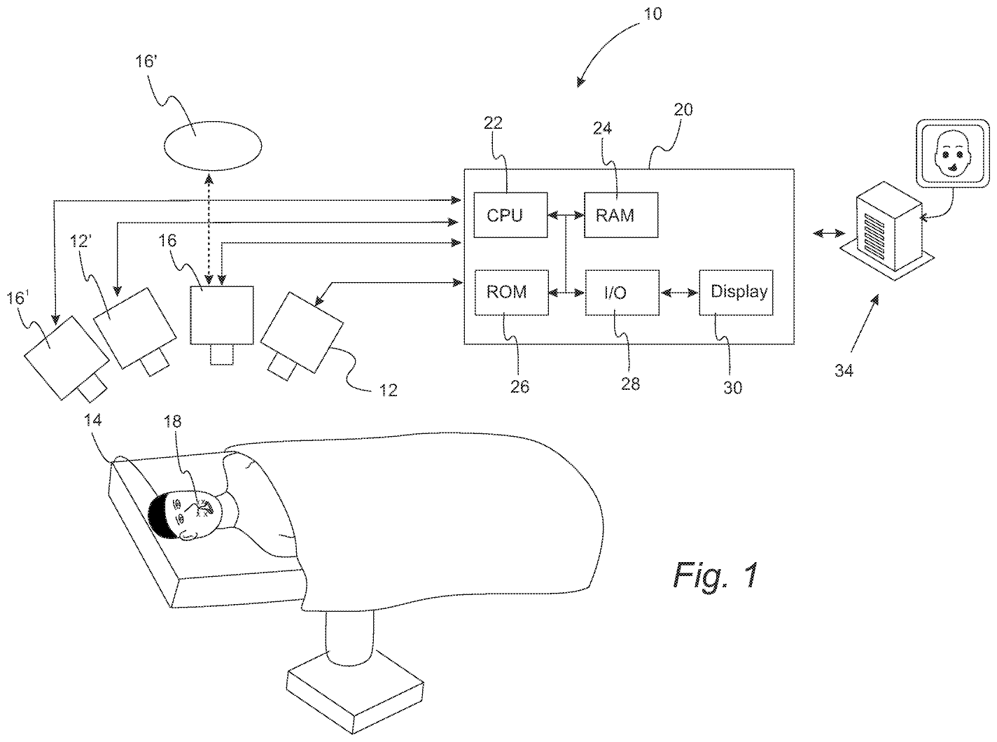

Referring to FIG. 1, a schematic of a system for guiding a surgical or medical procedure is provided. In particular, system 10 is used to guide surgical or medical procedures that rely on a detailed understanding of surface and underlying deep anatomy. Examples of such procedures include but are not limited to cleft lip surgery, ear reconstruction for microtia, cranial vault reconstruction for craniosynostosis, breast reconstruction after cancer resection, and flap-based reconstruction of traumatic and oncologic defects. System 10 is found to be particularly useful for guiding the surgical reconstruction of cleft lip. System 10 includes depth camera 12 for acquiring images and/or video from a subject 14 from a predetermined surgical site. In a refinement, system 10 can include one or more additional cameras 12′ which allow alternative views of the surgical site if a camera is obstructed. System 10 also includes projector 16 for projecting surgical markings or other surgical markings that guide surgical or medical procedures 18 onto subject 14 within the predetermined surgical site. In a refinement, the projector 16 projects the surgical markings onto a human surface. In another refinement, system 10 also includes one or more additional projector 16′ which allow alternative projection paths if a projector is obstructed. An example of a useful projector is the Insignia Portable Pico Projector Cube NS-PR166 (Insignia, Minneapolis, Minn.). The Insignia projector is a 2-inch cube that can be held in the palm of the hand and has the ability to project under standard lighting conditions. In addition, the projector can be mounted on a gooseneck and clamped onto an intravenous fluid pole for stability. In this context, the projector is a type of augmented reality system in which the surgical marking is projected onto human surfaces. In another refinement, the projections are integrated with wearable AR technologies such as AR glasses 16′.

System 10 also includes trained (and/or trainable) machine learning guide generator 20 is in electrical communication with depth camera 12 and projector 16. Machine learning guide generator 20 implements a trained model of the predetermined surgical site. In general, the trained machine learning guide generator is configured to interact with the projector to identify and place the surgical markings or other information that guides surgical or medical procedures onto the predetermined surgical site. In a refinement, the trained machine learning guide generator is configured to interact with the projector to identify machine-learned landmarks, bind these to a given subject, and project these landmarks and guides directly onto the predetermined surgical site. Typically, machine learning guide generator 20 is a computer system that executes the machine learning methods for determining and placing surgical markings that guide surgical or medical procedures. Trained machine learning guide generator 20 includes computer processor 22 in communication with random access memory 24. Computer processor 22 executes one or more machine learning procedures for the projection of surgical markings that guide surgical or medical procedures on a subject. Trained machine learning guide generator 20 also includes non-transitory memory 26 (e.g., DVD, ROM, hard drive, optical drive, etc.), which can have encoded instruction thereon for projecting the surgical markings that guide surgical or medical procedures using a machine learning algorithm. Typically, the machine learning instructions will be loaded into random access memory 24 from non-transitory memory 26 and then executed by the computer processor 22. Trained machine learning guide generator 20 also includes input/output interface 28 that can be connected to display 30, keyboard and mouse. When trained machine learning guide generator 20 has been trained as set forth below, surgical markings that guide surgical or medical procedures can be projected onto a subject during actual surgery. A surgeon can then make surgical cuts in soft tissue, bone, brain, etc. using the surgical marking as a guide. Advantageously, a user can preview surgical markings on a user interface rendered on display 30 before they are projected.

In a variation, trained machine learning guide generator 20 or another computing device is configured to bind a subject's anatomy to projected surgical markings such that the projections remain stable with movement of the subject. For example, one surface can be detected by the depth camera and read by an AI algorithm while another surface is a radiographic surface also read by AI algorithm. By binding these two surfaces using AI algorithms or other already established techniques, the internal anatomy is also bound (even with movement) and can therefore be accurately projected onto the patient's surface

In a variation, system 10 can include the combination of depth camera 12 and projector 16 operating as a structured light scanner which allows the creation of three-dimensional representations (i.e., three-dimensional digital images) of the surgical site. In this regard, the combination of the depth camera and the projection can acquire structured light scans. This three dimensional digital image can be digitally manipulated by a remote educator or expert using the device UI and these markings are then projected back onto the surface of the three-dimensional surgical site to help guide the procedure in real-time. These surgical markings or other information can be determined by machine learning guide generator 20 executing a trained AI algorithm (e.g., a trained neural network). In a refinement, a remote user can modify machine-generated markings and add additional surgical markings and other information. This remotely generated surgical information can be projected onto the subject during a surgical procedure in real-time. A remote user can interact with system view via remote user computing device 34 which can communicate with system 10 over a network, the Internet, or a cloud. Moreover, such a remote user can be a surgical expert. In a variation, a remote expert user interacting through a computing device 34 can assist in training other users for knowledge and skill transfer.

The creation of 3-dimensional representations (i.e., digital images) acquired by structured light scanning allows system 10 to be used dynamically during a surgical procedure. In such an application, the creation of 3-dimensional representations (i.e., digital images) of the live surgical site can be periodically updated with a new structured light scan. Each update allows application of various machine learning algorithms to further guide surgery as well as providing a new canvas for the remote expert to mark in order to further guide the procedure using the system's projection capabilities. This includes the identification of deep structures onto which the markings or other information can be projected. Each time a new layer of tissue becomes relevant to the surgical procedure, a new structured light scan can generate a new image that can be marked by the trained AI algorithm and/or a surgical expert (e.g., a remote surgical expert) for projection. Therefore, the trained AI algorithms can identify markings or other information to guide projection onto surfaces in the subject, including deep surfaces (e.g., under the skin).

In a variation, trained machine learning guide generator 20 is configured to guide each sequential step of the surgical or medical procedure by dynamically adjusting the surgical markings that guide surgical or medical procedures projected onto the subject 14 within the predetermined surgical site during the surgical or medical procedure. In particular, the projector can project deep anatomy onto the predetermined surgical site of the subject as well as directly onto deeper tissues to guide steps during surgery. In a refinement, surgical marking can be projected onto deep structures that are revealed during a surgical or medical procedure. In particular, the trained machine learning model allows the placement of surgical markings that guide surgical or medical procedures and is trained to place surgical markings in real-time regardless of the angle of the predetermined area. In this regard, the surgical markings are bound to the surface on which they are projected such that the projected marking moves as the subject moves remaining registered thereto.

In a refinement, a remote user (e.g., a surgeon) can interact with the surgical markings that guide surgical or medical procedures to make adjustments thereof. In another refinement, a remote operator can interact with a three-dimensional digital image of the predetermined surgical site and proposed surgical markings in order to add surgical guidance and/or make adjustments thereof. In another refinement, trained machine learning guide generator 20 is configured to acquire data during surgical or medical procedures to improve the accuracy of placing surgical markings that guide surgical or medical procedures for future surgical or medical procedures. It should be appreciated that system 10 allows remote guidance (with or without AI).

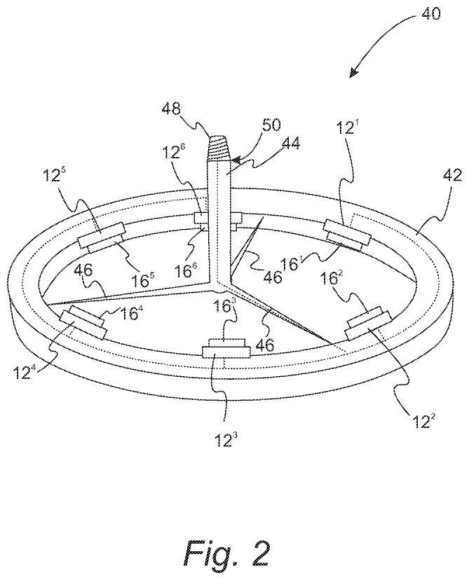

With reference to FIG. 2, a schematic of a projector-camera assembly for use with the system of FIG. 1 is provided. Projector-camera assembly 40 includes a plurality of cameras 12ⁱ and a plurality of projector 16^{j} mounted on a peripheral rim section 42. Label i is an integer label for the cameras running from 1 to imax which is the total number of cameras. Similarly, label j is an integer label for the cameras running from 1 to jmax which is the total number of projectors. Peripheral rim section 42 is attached to central support 44 (e.g., a hollow rod) typically by ribs 46. Electrical contact 48 is attached to end 50 of central support 44 and is adapted to screw into an operating room light fixture. Wiring 52 provide power to the cameras and projectors. This wire can run in the interior and/or along the exterior of central support 44, ribs 46, and peripheral rim section 42.

The machine learning algorithms executed by trained machine learning guide generator 20 will generally implement neural networks and, in particular, convolutional neural networks. In a variation, the trained neural network deployed is a high-resolution neural network. Many models generally downsample the dimensionality of the input at each layer in order to gain generalizability. However, in one refinement, the computer system 20 performs this down sampling of sample images in parallel with a series of convolutional layers that preserve dimensionality, allowing for intermediate representations with higher dimensionality.

In general, machine learning guide generator 20 is trained as follows in a two-phase training procedure. In the first training phase, the machine learning algorithm learns how to detect a plurality of anatomical features on digital images (e.g., photos from non-surgical subjects) to create a general detection (e.g., general facial detection) model. In a refinement, the general detection model is a point detection model. During this phase, a general detection model is created from a first set of annotated digital images from subjects. In the second phase, the general detection model is trained (e.g., by backpropagation) with a second set of annotated digital images from subjects that may or may not have the surgical condition being trained for. The annotations include a plurality of anthropometric surgical markings identified by an expert surgeon. In a refinement, before training the model, augmentation of the image dataset is implemented. This technique improves the robustness of the model and creates new training data from existing cleft images by generating mirror images of each picture. It should be appreciated that machine learning guide generator 20 can be trained in the same manner using 2-dimensional and 3-dimension image representations from a radiological imaging modality. In this situation, a set of 2-dimensional and 3-dimension image representations from subjects not having the condition needing surgery are annotated by a surgical expert for anatomical features to create the general detection model. In the second phase, the general detection model is trained (e.g., by backpropagation) with a second set of annotated two-dimensional and/or three-dimension digital image representations (i.e., three-dimensional digital images) from subjects that may or may not have the surgical condition being trained for. Again, the annotations include a plurality of anthropometric surgical markings identified by an expert surgeon.

In a refinement, the machine learning algorithm can be tested as follows. Each testing image is marked digitally by the expert surgeon, and automatically by the trained machine learning algorithm. The two-dimensional coordinates of each of the anatomic points generated by the machine learning algorithm (\tilde{ } x, \tilde{ } y) were compared to the two-dimensional coordinates of the human-marked points (x, y). The precision of each point was computed by calculating the Euclidean distance

$$d = \sqrt[2]{{( {x}_{i}^{k} - {x}_{i}^{∼ k} )}^{2} + {( {y}_{i}^{k} - {y}_{i}^{∼ k} )}^{2}}$$

between the human and AI-generated coordinates, normalized by dₙₒᵣₘ (for cleft lip this is intraocular distance, IOD) in order to standardize for image size.

$$Normalizad error = {δ}_{i}^{k} = \frac{d \{ ( {x}_{i}^{k} , {y}_{i}^{k} ) , ( {x}_{i}^{∼ k} , {y}_{i}^{∼ k} ) \}}{IOD} .$$

The superscript k indicates one of the landmarks and the subscript i is the image index. The normalized error for each point was averaged across the test cohort to obtain the normalized mean error (NME) for each anatomic point.

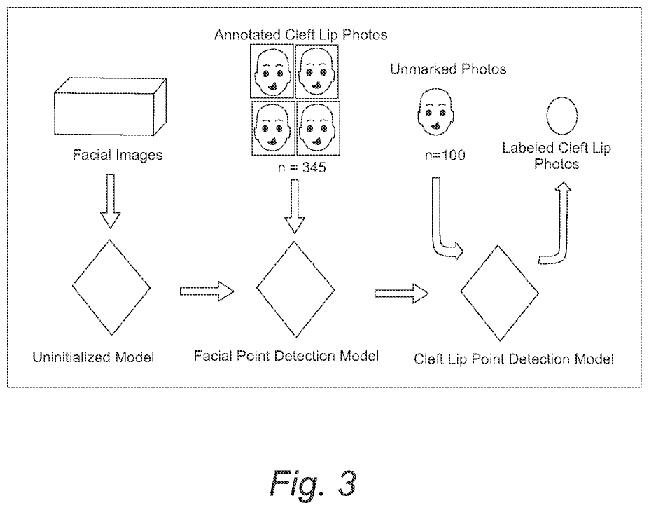

In accordance with this training method, a first set of annotated images of a predetermined area of a subject's surface is fed to a generic model. The images have annotated landmarks (e.g., lips, nose, and chin). Typically, the first set of annotated images includes thousands or hundreds of thousands of images. In the case of cleft lip, this first set of annotated images are images of faces. In a refinement, these first set of images are obtained from generic non-surgical patients. After training with the first set of annotated images, the model then becomes a generalized and robust single point detection model (e.g., a facial point detection model). Then the system is trained with a second set of annotated images annotated with surgical annotations for a surgical marking that guide surgical or medical procedures. The model can be tested with a set of unmarked images and verified by a surgical expert. In a variation, the model is trained to place the surgical markings in real-time regardless of the angle of the predetermined area. FIG. 3 outlines the training for the case of cleft lip.

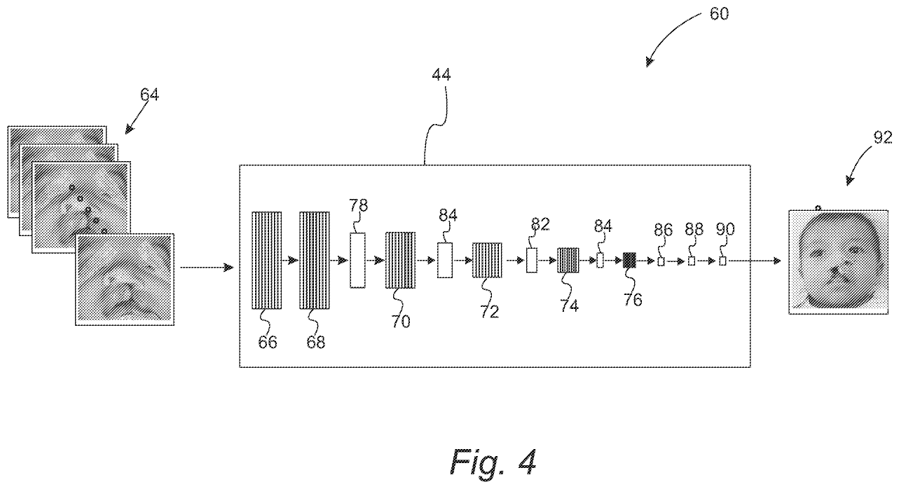

With reference to FIG. 4, an idealized schematic illustration of a convolutional neural network executed by trained machine learning guide generator 20 is provided. It should be appreciated that any deep convolutional neural network that operates on the pre-processed input can be utilized. The convolutional network 60 can include convolutional layers, pooling layers, fully connected layers, normalization layers, a global mean layer, and a batch-normalization layer. Batch normalization, a regularization technique, which may also lead to faster learning. Convolutional neural network layers can be characterized by sparse connectivity where each node in a convolutional layer receives input from only a subset of the nodes in the next lowest neural network layer. The convolutional neural network layers can have nodes that may or may not share weights with other nodes. In contrast, nodes in fully connected layers receive input from each node in the next lowest neural network layer. For both convolutional layer and fully connected layers each node calculated its output activation from its inputs, weight, and an optional bias.

During training, optimal values for the weight and bias are determined. For example, convolutional neural network 60 can be trained with a set of data 64 that includes a plurality of images that have been annotated (by hand) with surgical markings that guide surgical or medical procedures by an expert surgeon. As shown below, this approach has been successfully demonstrated for cleft lip. Convolutional neural network 60 include convolution layers 66, 68, 70, 72, 74, and 76 as well as pooling layers 78, 80, 82, 84, and 86. The pooling layers can be max pooling layer or a mean pooling layer. Another option is to use convolutional layers with a stride size greater than 1. FIG. 4 also depicts a network with global mean layer 88 and batch normalization layer 90. The present embodiment is not limited to by number of convolutional layers, pooling layers, fully connected layers, normalization layers, and sublayers therein.

Once trained, the neural network, and in particular, the trained neural network will receive images of a predetermined surgical site from a subject and then project surgical markings that guide surgical or medical procedures onto the predetermined surgical site in real-time as depicted by item 92.

With reference to FIG. 1, in another variation, the surface anatomy of the subject and his or her surface anatomy captured in digital radiological images are both processed by machine learning algorithms such that subject-specific radiological information is bound to that subject during a medical or surgical procedure. This binding of surface anatomy (patient and radiograph) can be accomplished with or without application of AI techniques. Digital radiologic images of deeper tissues (e.g. bone, brain, etc.) can be projected onto the predetermined surgical site of a subject 14 undergoing a surgical procedure in order to guide surgical decision-making. In another refinement, binding of a subject's surface anatomy with his or her surface anatomy as detected in a radiographic image allows for accurate detection and binding of deeper tissues as a surgery progresses. These deeper tissues (bone, brain, artery, etc.) are accurately identified so that new machine learning algorithms can detect their three-dimensional structure and enable surgically oriented projection directly onto these deeper tissues. In another refinement, the machine learning algorithm can be trained to identify key anatomical structures and features of the imaging (e.g., bone, blood vessel, nerve, tendon) and generate surgical markings/plans that either incorporate or circumvent these key anatomical features (e.g. mark flap based on vascularity, avoid injury to important nerve branches, etc.). Then projector 16 can project the surgical markings/plan onto the subject and lock onto these key anatomical features. Examples of radiological imaging techniques that can be used include but are not limited to, CT scans, MRI, X-ray, ultrasound, angiograph, and the like. In a refinement, machine learned algorithms can identify angiosomes, relaxed skin tension lines, and the like—enabling the projector to illuminate this information directly on the patient's surface.

In one variation, the trained machine learning guide generator 20 or another computing device is configured to receive and store diagnostic image data such that images are generated from the image data onto the predetermined surgical site relevant to a surgical procedure. In a refinement, the trained machine learning guide generator 20 or another computing device is configured to receive and store subject-specific radiologic image data. Advantageously, the surface anatomy that the system for guiding a surgical or medical procedure can be matched to the surface anatomy of the radiological image scan to bind anatomy. In a refinement, surface anatomy of patient can be matched to the deep anatomy delineated by radiological images.

Key anthropometric markings (or other anatomical landmarks) can be detected by the machine learning algorithm to guide this binding. Once bound, the projector 16 can project a subject's deep anatomy onto the surface (e.g., such as a facial bone fracture). In this context, deep anatomy means anatomical structures below the subject's skin. In one refinement, the surface anatomy is not pathological, but the underlying anatomy is, that the normal surface anatomy on radiology is bound to the normal surface that our cameras read in order to bind these surfaces and accurately guide the projection of abnormal bones underneath. Since the trained machine learning algorithms that also recognize abnormal surface anatomy (such as cleft lip), we could use AI to bind abnormal surface anatomy on radiology and on a patient to project their normal or abnormal underlying structures onto the surface. For example, children with cleft lip often have an abnormal underlying bone of the jaw and cartilage of the nose and these can be projected onto their surface to guide surgical planning or patient consultation.

In one example, a surgery is conducted where abdominal tissue and its vascular supply are dissected in order to reconstruct the breast. The preoperative imaging scan (e.g., a CT scan with intravenous contrast) is provided to feed into system 10. The machine learning algorithm can then plan the incisions of the abdominal tissue to incorporate the major blood vessel (e.g., determined by CT scan). The projection will include the planned incision in addition to the projection of the course of the blood vessel (determined by the CT scan). Finally, the projection will also lock onto the external anatomy (i.e., boney prominence such as but not limited to the anterior superior iliac spine, pubis, inferior ribs) to keep the projection locked and in sync during patient movement. In another example, the surgical goal is to reconstruct a bony defect of the orbital floor with a plate and screws. The preoperative CT scan of the face can feed into the machine learning algorithm. The machine learning algorithm then detects the shape of the contralateral non-traumatized orbit and projects a mirrored version onto the traumatized side, illuminating how the surgeon can shape the plate to reconstruct the bony defect. This projection will also lock onto the external surface anatomy to bind the projection and keep it stable and in coordination with patient movement.

In a variation, machine learning guide generator 20 presents a user interface on display 26. This user interface includes a main screen that calls for login information such as username and password. One or more operational interface can then be presented with the following features:
Capture operative area: structured light scan creates a 3d digital representation of an area that will be used by a computer to place AI-generated information and connect the remote expert to the projection capability of device 10 using his/her UI.
Preview surgical markings: displays surgical markings on UI but does not project onto the patient.
Project surgical markings: displays previewed surgical markings onto the patient
Stop projection: Stops projecting an image onto the patient
Freeze surgical marking in current position→Locks surgical marking in place so the surgeon cannot move surgical markings with gestures or UI (computer)
Unfreeze surgical markings→Surgeon able to move surgical markings with surgical markings on UI (computer) or via finger gesture on the patient.
Operative tools:
   a. Marking Tools (Images)
      1. Arrow (up, down, left, and right)—need all four of these
      2. Pen image
      3. Asterix
      4. Scalpel image
      5. Stop sign
      6. Retractor
      7. Point (same size as surgical markings generated by AI)
Record Surgery: records everything happening during the projection phase onwards
Screenshot: Takes an image of the operative area as surgeon modifies projected plan live.
Save case: Saves case for the operator so they can refer back to their case (must have patient identifiers→Medical record number (MRN), Date of birth, name, procedure.
   It should be appreciated that all patient protected information is encrypted in a HIPAA compliant platform

The following examples illustrate the various embodiments of the present invention. Those skilled in the art will recognize many variations that are within the spirit of the present invention and scope of the claims.

### Materials and Methods

In cleft lip surgery, surgical experts require years of training before operating independently. In order to design a cleft lip repair, anthropometric landmarks of the cleft lip and nose must be accurately identified and marked. Using these landmarks, several variations in surgical design are possible based on surgeon preference and experience. Because of the anatomic complexity and three dimensionalities of this small area of surface anatomy (<10 sq cm), as well as global need for teaching cleft lip surgery in under-resourced regions, we chose to use this condition as a proof of concept for machine learning assisted surgical planning and guidance. While this is one example of the system for guiding a surgical or medical procedure set forth above, it is important to note that the technology can be used in a broad array of surgical or medical procedures that rely heavily on a detailed understanding of anatomy (e.g. ear reconstruction for microtia, cranial vault reconstruction for craniosynostosis, breast reconstruction after cancer resection, reconstruction of traumatic or oncologic defects)

A High-Resolution Net (HR-Net) architecture was adopted to develop the AI model for the placement of cleft anthropometric markings. HR-Net is a recent family of CNN-based deep learning architecture specialized in computer-vision tasks is adapted. This architecture has previously been used as the backbone to accomplish tasks such as object detection, image classification, pose estimation, and even facial landmark detection. A limitation of these networks is the requirement of large data sets needed to train the algorithm. Given the difficulty in acquiring such quantities of cleft lip images, transfer learning” in which the machine learning algorithm learns how to detect some anthropometric markings on non-cleft photos to create a general facial detection model is utilized. The model is then trained with cleft images with digitally marked anthropometric landmarks. Before training the model, the standard practice of “augmenting” our dataset is implemented. This technique improves the robustness of the model and creates new training data from existing cleft images by generating mirror images of each picture.

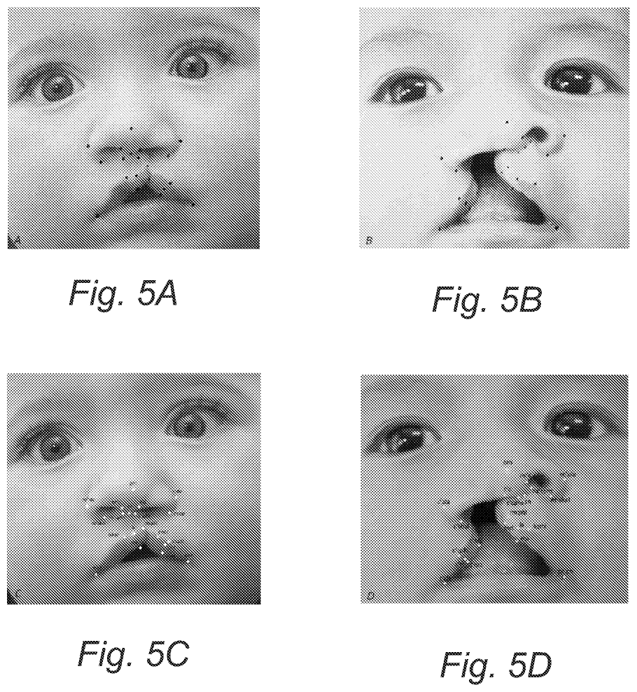

To select the appropriate sample size needed to train and test the model, experiments using existing facial recognition algorithms were run. The Normalized Mean Error (NME) for training and testing cohorts is generally found to converge at around 300 images with minimal additional decreases in error appreciated with even four times this number. Therefore, 345 two-dimensional photos of infants and children with unilateral cleft lip were utilized to train were utilized to develop and test the AI cleft model. The aggregate images were divided into those used for training (80%) and those for testing (20%). At the supervision of an expert cleft surgeon, training images were individually annotated for 21 well-established cleft anthropometric landmarks and points important during surgical design (FIGS. 5A and B). These hand-marked points were digitized and used to train the cleft AI algorithm. Nearly all images were provided by Global Smile Foundation, a not-for-profit international cleft outreach organization. Photos taken were of the full face and from a range of angles (frontal to submental). Informed consent was obtained from all individual participants or their parents for photography, use of their images in research and abided by the guidelines in the Declaration of Helsinki. A broad range of patient ethnicity (Hispanic (n=245), African (n=65), Middle Eastern (n=35)), Gender (Male (n=241), Female (n=104)), and type of cleft (Left sided cleft lip (n=206), Right sided cleft lip (n=139), Complete (n=312), Incomplete (n=33). Individual photos were not associated with numeric age; however, a broad range of ages was represented upon review of the photographs (infant to adult).

The AI algorithm was tested as follows. Each testing image was marked digitally by the expert cleft surgeon, and automatically by the cleft AI algorithm. The two-dimensional coordinates of each of the 21 anatomic points generated by the AI algorithm (\tilde{ } x, \tilde{ } y) were compared to the two-dimensional coordinates of the human-marked points (x, y). The precision of each point was computed by calculating the Euclidean distance

$$d = \sqrt[2]{{( {x}_{i}^{k} - {x}_{i}^{∼ k} )}^{2} + {( {y}_{i}^{k} - {y}_{i}^{∼ k} )}^{2}}$$

between the human and AI-generated coordinates, normalized by dₙₒᵣₘ (intraocular distance, IOD) in order to standardize for image size.

$$Normalizad error = {δ}_{i}^{k} = \frac{d \{ ( {x}_{i}^{k} , {y}_{i}^{k} ) , ( {x}_{i}^{∼ k} , {y}_{i}^{∼ k} ) \}}{IOD} .$$

The superscript k indicates one of the landmarks and the subscript i is the image index. The normalized error for each point was averaged across the test cohort to obtain the normalized mean error (NME) for each anatomic point.

### Results

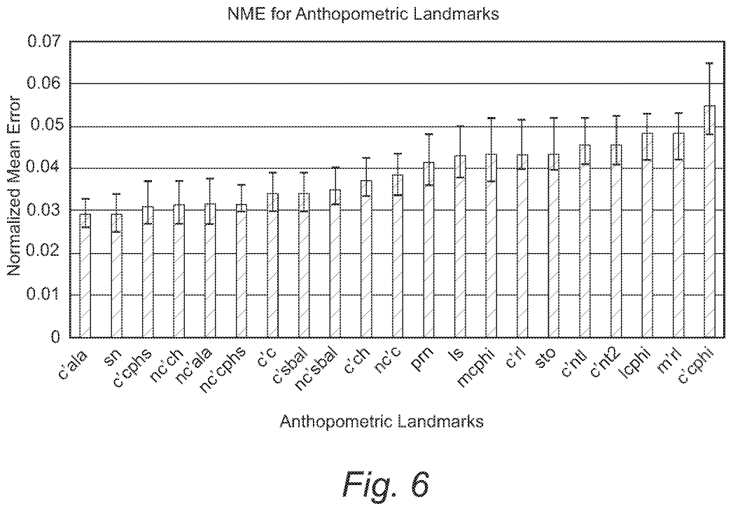

The cleft AI model was trained to recognize and mark 21 anatomic points representing important anthropometric landmarks for understanding cleft nasolabial anatomy and for designing various types of nasolabial repair. For each point, the NME was calculated and is represented in FIG. 6. All NME values were between 0.029 and 0.055. The largest NME was for cleft-side cphi point (Cupid's bow peak). The smallest NME value was for cleft-side alare point. Our cleft AI model can mark 2D photos and video of patients with a range of cleft lip/nose severity (FIGS. 5A and 5B). Additionally, the cleft AI model can identify cleft nasolabial landmarks over a wide range of viewing angles (video available—demonstrates AI algorithm at work in the operating room over a wide range of viewing angles for a child with unilateral cleft lip)

The system for guiding a surgical or medical procedure is able to successfully detect and digitally place surgical markings that guide surgical or medical procedures onto the operative field. Our system's cameras capture an image of the subject's cleft lip and 21 machine-learned points are identified and bound to this subject-specific surface. With these points bound using the machine learned algorithm, the projector can project these key landmarks onto the surface of the image (video available—demonstrates 21 landmarks being detected on an image of a child with cleft lip by depth camera with landmarks bound by machine-learned compute device and then projected back onto image of child with cleft lip). Binding of these machine-learned points by our artificial intelligence algorithm also allows for accurate projection when the subject moves (video available—demonstrates 21 landmarks quickly and accurately adjusting to new position of image of child with cleft lip). Since 2D images projected onto a 3D landscape can cause distortion, projection of the surgical markings that guide surgical or medical procedures onto a patient body needs to account for each individual's complex topography. This can be accomplished by applying depth-adjustment software to the machine learning algorithms. With this understanding the computing system can perform digital manipulations to outgoing projections, augmenting them to more accurately project onto 3D surfaces.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

Sayadi et al. A Novel Innovation for Surgical Flap Markings Using Projected Stencils. DOI: 10.1097/PRS.0000000000004708
Vyas et al. Using Virtual Augmented Reality to Remotely Proctor Overseas Surgical Outreach: Building Long-Term International Capacity and Sustainability. DOI: 10.1097/PRS.0000000000007293
Sayadi L, Hamdan U, Hu J, Zhangli Q, Vyas R. Harnessing the Power of Artificial Intelligence to Teach Cleft Lip Surgery. Plastic and Reconstructive Surgery Global Open Journal. June 2022.

What is claimed is:

1. A system for guiding a surgical or medical procedure, the system comprising:
a depth camera for acquiring images and/or video from a predetermined surgical site of a subject during the surgical or medical procedure;
a projector for projecting markings and/or remote guidance markings onto the predetermined surgical site during the surgical or medical procedure such that these markings and guides enhance procedural decision-making; and
a trained machine learning guide generator in electrical communication with the depth camera and the projector, the trained machine learning guide generator implementing a trained machine learning model specific to the predetermined surgical site, the trained machine learning guide generator configured to control the projector using the trained machine learning model such that surgical markings are projected onto the subject.

2. The system of claim 1, wherein the projector projects the surgical markings or other surgical information onto the predetermined surgical site.

3. The system of claim 1, a combination of the depth camera and the projector cooperate to operate as a structured light scanner for creating three-dimensional digital images of the predetermined surgical site or another area relevant to a surgical procedure.

4. The system of claim 1 further comprising a remote user computing device configured to allow a remote user to interact with the system and the three-dimensional digital image of the predetermined surgical site.

5. The system of claim 1, wherein the surgical or medical procedure is cleft lip surgery, ear reconstruction for microtia, cranial vault reconstruction for craniosynostosis, breast reconstruction after cancer resection, or reconstruction of traumatic or oncologic defects.

6. The system of claim 1, the trained machine learning guide generator or another computing device is configured to bind a subject's anatomy to projected surgical markings such that projections remain stable with movement of the subject.

7. The system of claim 1, wherein the trained machine learning guide generator or another computing device includes a machine learning algorithm trained to identify anatomical structures identified by radiological imaging techniques.

8. The system of claim 1, wherein the trained machine learning guide generator or another computing device is configured to bind surface anatomy captured by the depth camera to surface anatomy captured on radiographs such that applying machine learning algorithms to each identifies locations of shared surface landmarks with images of normal or pathologic underlying anatomic structures projected onto a surface of the predetermined surgical site.

9. The system of claim 1, wherein the surgical or medical procedure is cleft lip surgery.

10. The system of claim 1 wherein the trained machine learning guide generator is configured to guide sequential steps of the surgical or medical procedure by dynamically adjusting the surgical markings projected onto the subject during the surgical or medical procedure.

11. The system of claim 1 wherein the trained machine learning model allows placement of the surgical markings in real-time regardless of an angle of the predetermined surgical site.

12. The system of claim 1 wherein the trained machine learning guide generator is configured to interact with the projector to identify machine-learned landmarks, bind these to a given subject, and project these landmarks and guides directly onto the predetermined surgical site.

13. The system of claim 1 wherein a remote operator can interact with a three-dimensional digital image of the predetermined surgical site and propose surgical markings in order to add surgical guidance and/or make adjustments thereof.

14. The system of claim 1 wherein the trained machine learning guide generator is configured to acquire data during surgical or medical procedures to improve accuracy of placing the surgical markings for future surgical or medical procedures.

15. The system of claim 1 wherein the trained machine learning guide generator executes one or more neural networks.

16. The system of claim 15 wherein the trained machine learning guide generator executes one or more convolutional neural networks.

17. The system of claim 15 wherein the trained machine learning guide generator executes a high-resolution neural network.

18. The system of claim 17 wherein the trained machine learning guide generator is configured to down sample images in parallel with a series of convolutional layers that preserve dimensionality, allowing for intermediate representations with higher dimensionality.

19. The system of claim 17 wherein the trained machine learning guide generator is trained by:
providing a first set of annotated images of a predetermined area of a subject's surface to a generic model to form a point detection model; and
training the trained machine learning guide generator using the point detection model with a second set of annotated images annotated with surgical annotation for each surgical marking.

20. The system of claim 1, wherein the trained machine learning guide generator or another computing device is configured to receive and store subject-specific radiologic image data.

21. The system of claim 20, wherein the trained machine learning guide generator is further trained to identify anatomical structures from radiographic imaging modalities. By using machine learning algorithms to bind a subject's surface anatomy to its radiographic correlate, radiologic images of underlying structures can be projected onto the predetermined surgical site.

22. The system of claim 20, wherein the projector projects deep anatomy onto the predetermined surgical site of the subject as well as directly onto deeper tissues to guide steps during surgery.

23. A method for guiding a surgical or medical procedure, the method comprising:
acquiring images and/or video from a predetermined surgical site of a subject during the surgical or medical procedure; and
projecting surgical markings that guide surgical or medical procedures onto the predetermined surgical site during the surgical or medical procedure, wherein positions of the surgical markings are determined by a trained machine learning guide generator.

24. The method of claim 23, wherein the surgical or medical procedure is cleft lip surgery, ear reconstruction for microtia, cranial vault reconstruction for craniosynostosis, breast reconstruction after cancer resection, or reconstruction of traumatic or oncologic defects.

25. The method of claim 23, wherein the trained machine learning guide generator is configured to guide each step of the surgical or medical procedure by dynamically adjusting the surgical markings projected onto the subject during the surgical or medical procedure.

26. The method of claim 23, wherein the trained machine learning guide generator is configured to acquire data during surgical or medical procedures to improve accuracy of placing surgical markings for future surgical or medical procedures.

27. The method of claim 23, wherein the trained machine learning guide generator executes one or more neural networks.

28. The method of claim 23 wherein the trained machine learning guide generator is configured to down sample images in parallel with a series of convolutional layers that preserve dimensionality, allowing for intermediate representations with higher dimensionality.

29. The method of claim 23, wherein the trained machine learning guide generator or another computing device is configured to receive and store diagnostic image data such that images are generated from the diagnostic image data are projected onto the predetermined surgical site of the subject.

30. The method of claim 23, further comprising binding a subject's anatomy to projected surgical markings such that projections remain stable with movement of the subject.

31. The method of claim 30, wherein a machine learning algorithm is trained to identify anatomical structures identified by radiological imaging techniques such that images of the anatomical structures are projected along with the surgical markings onto the predetermined surgical site of the subject.

32. The method of claim 31, wherein radiological imaging techniques includes CT scan, MRI, ultrasound, and/or angiography.

33. The method of claim 23, wherein a subject's deep anatomy is projected onto the predetermined surgical site of the subject.

34. A system for guiding a surgical or medical procedure, the system comprising:
a depth camera for acquiring images and/or video from a predetermined surgical site of a subject during the surgical or medical procedure;
a projector for projecting surgical markings that guide surgical or medical procedures onto predetermined surgical site during the surgical or medical procedure; and
a trained machine learning guide generator in electrical communication with the depth camera and the projector, the trained machine learning guide generator implementing a trained machine learning model for the predetermined surgical site, the trained machine learning guide generator configured to control the projector using the trained machine learning model such that surgical markings are projected onto the subject, the trained machine learning model being trained by:
creating a general detection model from a first set of annotated digital images, each annotated digital image being marked or annotated a plurality of anatomic features; and
training the general detection model by backpropagation with a second set of annotated digital images from subjects that are surgical candidates, each digital images including a plurality of anthropometric markings identified by an expert surgeon.

35. A system for guiding a surgical or medical procedure, the system comprising:
a depth camera for acquiring images and/or video from a predetermined surgical site of a subject during the surgical or medical procedure; and
a projector for projecting markings and/or remote guidance markings onto the predetermined surgical site during the surgical or medical procedure such that these markings and guidance markings enhance procedural decision-making.
